(19) United States
(12) Patent Application Publication
(10) Pub. No.: US 2009/0240439 A1
(43) Pub. Date: Sep. 24, 2009

(54) Predicting responses to drugs or drug cadidates

(75) Inventors: Alan George Clark, West Lothian (GB); Steven Howell, Perhshire (GB); Piet Hugo Christiaan Moerman, Martens-Latem (BE)

Correspondence Address: Steptoe & Johnson LLP, 1330 Connecticut Avenue, NW, Washington DC, DC, 20036, US

(73) Assignee: Inverness Medical Switzerland GMBH, Zug (CH)

(21) Appl. No.: 11/990,698

(22) PCT Filed: Aug. 29, 2006
(86) PCT No.: PCT/IB2006/002352

§ 371 Date: Apr. 28, 2008

Related U.S. Application Data
(60) Provisional application No. 60/712,393, filed on Aug. 31, 2005.

(51) Int. Cl. G06F 19/00; G01N 33/53

(52) U.S. Cl. 702/19; 436/501

(57) ABSTRACT

Patients can be segregated into groups expected to have differential responses to drug treatment based on test results. Patient sub-populations expected to benefit from a particular treatment can thus be identified and directed to that treatment. Similarly, sub-populations expected to suffer a greater risk of side effects from a particular treatment can be identified and steered to other, safer treatments. A patient under treatment can be monitored for safety.

### TECHNICAL FIELD

This invention relates to prediction of responses to drugs or drug candidates.

### BACKGROUND

Clinical testing of drug candidates typically includes testing for safety and efficacy of the drug. One component of safety testing is review of patient reports of adverse events after the patients have taken the drug. Sometimes drugs are initially deemed to be safe based on the results of safety testing, only to have the safety of the drug called into question later. For example, a side effect related to the drug may not be discovered during testing when the effect occurs only in a subset of patients, for example, those patients who are at risk for suffering the side effect.

### SUMMARY

Test results can predict patient responses to drugs. When a correlation is shown between a test result and a patient response, that information can be used to categorize patients. Categorizing patients can be useful in many situations, including during drug development.

In one aspect, a method of predicting patient response to a drug or drug candidate includes obtaining a plurality of test results for a plurality of patients. The plurality of test results includes, for each patient, at least one result of a biochemical test, a medical test, or a genetic test; and at least one result of a test of patient response to the drug or drug candidate. The method includes detecting a correlation between the result of a biochemical test, a medical test, or a genetic test and the result of the test of patient response to the drug.

The test of patient response to the drug or drug candidate can a test of drug efficacy or of drug safety. The plurality of test results can include, for each patient, at least one result of a plurality of biochemical tests. The plurality of test results can include, for each patient, a plurality of results of a plurality of biochemical tests. At least one biochemical test can include an immunoassay.

The method can include obtaining at least one result of a biochemical test, a medical test, or a genetic test, from a prospective patient; and predicting a response of the prospective patient based on the result obtained for the prospective patient and the detected correlation. Obtaining at least one result from the prospective patient can include obtaining at least one result of a plurality of biochemical tests from the prospective patient.

In another aspect, a method of monitoring a patient includes administering a drug or drug candidate to a patient, obtaining at least one result of a biochemical test, a medical test, or a genetic test from the patient, and correlating at least one result with at least one measure of patient response.

In another aspect, a method includes obtaining a result of at least one of a biochemical test, a medical test, and a genetic test performed on a human subject, referencing a correlation between the result of the at least one biochemical test, medical test, and genetic test and a human response to a drug, the correlation having been determined based on a result of the at least one biochemical test, medical test, and genetic test obtained from each of multiple reference human subjects and a response of each of the multiple human subjects to a drug, and predicting a response of the human subject to the drug based on the result of the at least one biochemical test, medical test, and genetic test and human response and the correlation.

The measure of patient response is a measure of drug or drug candidate efficacy or of drug of drug candidate safety. Obtaining at least one result from the patient can include obtaining at least one result of a plurality of biochemical tests from the patient. Obtaining at least one result from the patient can include includes obtaining a plurality of results of a plurality of biochemical tests from the patient. At least one result of the plurality of biochemical tests can correlates with a measure of drug or drug candidate efficacy, or drug or drug candidate safety. The method can include altering the administering of the drug or drug candidate to the patient based on the obtained result.

The drug or drug candidate can be a statin. Obtaining at least one result can include obtaining a result of a test for a marker of endothelial dysfunction, a marker of inflammation, a marker of plaque stability, a marker of systolic function, a marker of safety, or a combination thereof.

Biochemical tests that can be used include one or more tests for a biomarker, for example, urotensin II, urotensin related peptide, prourotensin II, prourotensin related peptide, oxidized low-density lipoprotein (ox-LDL), malondialdehyde-modified LDL (MDA-LDL), oxygen regulated protein 150 (ORPl50, also known as hypoxia upregulated 1 (Hyou1), or Cab140), relaxin (also known as RLX), soluble CD4OLigand/TRAP (sCD40L), C-reactive protein (hsCRP), interleukin-6 (IL6, also known as interferon Beta-2 (IFNB2), B-cell differentiation factor, B-cell stimulatory factor 2 (BSF2), hepatocyte stimulatory factor (HSF), hybridoma growth factor (HGF)), placental growth factor (PGF), soluble P-selectin (also known as P-selectin, sP-Selectin), medroxyprogesterone acetate (MPA), soluble fibrin (also known as s-Fibrin, SF), matrix metalloproteinase 9 (MMP-9, also known as GELB, CLG4B, gelatinase B, 92 kDa gelatinase, 92 kDa type IV collagenase), myeloperoxidase (MPO), asymmetric dimethylarginine (ADMA), lipoproteill phospholipase A2 (Lp-PLA2), pregnancy-associated plasma protein-A (P-APPA), proteinase activated receptor 1 (PAR-1), thrombin receptor Coagulation factor II receptor (CF2R, TR), poly-ADP ribose polymerase (P-PARP), Troponin (for example, Troponin-I), CK-MB, myoglobin, Nourin-1, ischemia modified albumin (IMA), BNP/Pro-BNP, isoprostanes, uroguanylin, prouroguanylin, cardiolipin, cathepsin, cystatin C, cytochrome c, Fas and Fas ligand (also known as CD95, APO-1), Choline, Caspase-1 (also known as interluekin converting enzyme (ICE)), Creatinine, Atrial Natriuretic Peptide (ANP/N-ANP), or osteoprotegerin (also known as OPG/OCIF).

The details of one or more embodiments are set forth in the drawings and description below. Other features, objects, and advantages will be apparent from the description, the drawings, and from the claims.

### BRIEF DESCRIPTION OF THE DRAWING

FIG. 1 is diagram illustrating a diagnostic device and an associated testing cartridge.

### DETAILED DESCRIPTION

In general, patients respond heterogeneously to a medication. Equivalent doses of the same drug can act differently in different people, both in therapeutic effect and side effects. A drug can have side effects that can have serious consequences for a subset of patients who take the drug. As a result, drugs that are beneficial for many patients can be withdrawn from the market because of safety considerations, since there is no way to determine who is susceptible to the side effects. Similarly, a subset of patients may enjoy a stronger benefit from a drug than other patients.

A test can provide data that predicts a patient's probable response to a drug. The test can be, for example, a genetic test, a biochemical test, or a medical test. A genetic test can include identification of single nucleotide polymorphisms in a patient. A biochemical test can include determining a level of a biomarker such as, for example, a protein, a mono- or polysaccharide, a lipid, a nucleic acid, a metabolite, a hormone, or other biochemical, in a patient. A medical test can include, for example, a medical imaging test (such as an X-ray, MRI, intravascular ultrasound, or other imaging test), blood pressure, or electrocardiogram.

When data from tests is compared to patient outcomes after therapy, correlations can be revealed. For example, one group of patients having a high level of a biomarker may have shown a strong response to a particular drug during clinical trials, whereas patients with a low level of the biomarker showed a weak response. Some correlations can involve results of more than one test. Once a correlation has been found, future patients can be segregated into groups (e.g., a strong-response group and a weak-response group) based on the results of their tests. The tests can be useful in determining which patients are likely to benefit from a particular treatment, and in determining which patients are likely to suffer side effects from the treatment. The latter can be particularly important when the side effects are serious or life-threatening. Desirably, a small number (e.g., less than 100, less than 50, less than 20, less than 10, less than 5, or fewer) of tests can be identified that strongly predict a patient's response to a drug. More preferably, those tests can be administered by a simple inexpensive device. In some cases, the device can be suitable for point-of-care use by a health care provider, or for in home use by a patient.

Knowledge about the mechanism of action of a particular drug may suggest tests that are likely to correlate with patient response. For example, if a drug is known to alter a particular metabolic pathway, correlations between enzyme levels or metabolite levels on the pathway could be sought. Alternatively, a genetic test might reveal that particular SNPs in coding regions of genes encoding the enzymes on the pathway correlate with a patient's response. In some cases, a biochemical or a medical test will be desirable, as genetic tests cannot reveal environmental effects on a patient's response.

Biomarkers can include, but are not limited to, markers of myocardial stretch, myocardial apoptosis or injury, myocardial ischemia, anemia, renal function, electrolytes, and markers of sodium balance.

Patient tests are frequently performed in conjunction with a clinical trial (e.g., a phase 1, phase 2, phase 3 or phase 4 clinical trial). Because patients in clinical trials are closely monitored to determine both safety and efficacy of a drug, these test results are a rich source of data for finding correlations between test results and patient outcome. Searches for correlations can be simultaneously with the clinical trial, for example to seek clinical validation of a hypothesized correlation. A search can also be done retrospectively, for example by data mining of stored results and outcomes, or by conducting new tests on patient samples collected during the trial and stored.

For example, a drug which has stalled in clinical development due to ambiguous efficacy may have no apparent safety issues. In such a case, the ability to identify a patient population that will respond to the drug can rescue the stalled development. Retrospective analysis of clinical trial records can reveal correlations between test results and drug response. Based on the correlation, a companion diagnostic test can partition responders and non-responders, for example by measuring of a biomarker. This can provide a clear demonstration of drug efficacy in a subset of patients, and allow progression of drug development. In this way, institutions that have spent large sums of money developing a safe drug only to see questions about efficacy hurt sales can potentially create new, targeted markets for the drug. The U.S. Food and Drug Administration (FDA) is considering the regulatory framework that might be applied to such drug-diagnostic combinations (see, for example, “Drug-Diagnostic Co-Development Concept Paper”, FDA, April 2005, which is incorporated by reference in its entirety), indicating that it expects for such combinations to be used commonly.

Physicians can use test results to help guide treatment decisions. For example, a level of oxidized LDLs can be helpful in deciding whether to prescribe a statin to a patient. Higher levels of oxidized LDLs will favor the use of a statin, because statins have been shown to have an antioxidant activity in addition to LDL-lowering activity. A level of soluble CD40 can aid in deciding whether to give a GPIIbIIIa inhibitor. A BNP measurement, or an ischemia-modified albumin (IMA) measurement, can indicate the desirability of treating heart failure with a vasodilator as opposed to diuretics. A free fatty acid test can indicate whether or not a PPAR agonist (such as, for example, rosiglitazone) should be given. A BNP test can also suggest the need for ACE inhibitor treatment in heart failure. When an ACE inhibitor is given, the patient can be further tested for creatinine levels, to monitor side effects on kidney function.

As an example, patients who are candidates for HMG-CoA reductase inhibitor treatment (i.e., statin treatment) can be tested to follow therapeutic efficacy and safety, in order to increase patient compliance. A personal monitoring system can measure both positive indicators of therapeutic effectiveness, and markers indicating a clear safety profile. The tests administered by the system can include tests of markers of endothelial dysfunction, inflammation, plaque stability, systolic function and markers demonstrating safety.

One safety issue of particular concern for patients taking statins is rhabdomyolysis. Rhabdomyolysis is a disorder which affects the integrity of the sarcolemma of skeletal muscle. This results in the release of potentially toxic biomarkers into the circulation. This in turn can result in potentially fatal complications including myoglobinuric acute renal failure, hyperkalaemia and cardiac arrest.

Myalgias and muscle weakness are the most common presenting symptoms in patients. The physician must be alert to the diagnosis of rhabdomyolysis and to its subtle presentation to prevent the most severe complication of acute renal failure. Most cases of rhabdomyolysis are due to trauma, alcohol abuse, and drug abuse but acquired rhabdomyolysis has been linked with the administration of statins (See, for example, “Rhabdomyolysis with statins” http://www.jr2.ox.ac.uk/bandolier/band131/b131-2.html, which is incorporated by reference in its entirety).

The primary diagnostic indicator of rhabdomyolysis is an elevated serum creatine phosphokinase (CK) to at least five times the normal value. This elevated level of CK excludes myocardial infarction and other causes. The CK-MM isoenzyme predominates in rhabdomyolysis, comprising at least 98% of the total value.

A device for testing patients who are taking statins can measure levels of a marker of inflammation such as C-reactive protein (CRP), a marker of endothelial function such as oxidised LDL, a marker of plaque stability such as matrix metalloproteinase 9 (MMP-9) and a marker of systolic function such as brain natriuretic peptide (BNP) or NT-proBNP, a marker of rhabdomyolysis such as CK or skeletal troponin I, or a combination thereof. In some embodiments, the device is configured to measure each marker in a single sample, such as a blood sample obtained by finger stick.

Biomarkers than can be tested include, but are not limited to: urotensin II, urotensin related peptide, prourotensin II, prourotensin related peptide, oxidized low-density lipoprotein (ox-LDL), malondialdehyde-modified LDL (MDA-LDL), oxygen regulated protein 150 (ORP150, also known as hypoxia upregulated 1 (Hyou1), or Cab140), relaxin (also known as RLX), soluble CD40Ligand/TRAP (sCD40L), C-reactive protein (hsCRP), interleukin-6 (IL6, also known as interferon Beta-2 (IFNB2), B-cell differentiation factor, B-cell stimulatory factor 2 (BSF2), hepatocyte stimulatory factor (HSF), hybridoma growth factor (HGF)), placental growth factor (PGF), soluble P-selectin (also known as P-selectin, sP-Selectin), medroxyprogesterone acetate (MPA), soluble fibrin (also known as s-Fibrin, SF), matrix metalloproteinase 9 (MMP-9, also known as GELB, CLG4B, gelatinase B, 92 kDa gelatinase, 92 kDa type IV collagenase), myeloperoxidase (MPO), asymmetric dimethylarginine (ADMA), lipoprotein phospholipase A2 (Lp-PLA2), pregnancy-associated plasma protein-A (P-APPA), proteinase activated receptor 1 (PAR-1), thrombin receptor Coagulation factor II receptor (CF2R, TR), poly-ADP ribose polymerase (P-PARP), Troponin (for example, Troponin-I), CK-MB, myoglobin, Nourin-1, ischemia modified albumin (IMA), BNP/Pro-BNP, isoprostanes, uroguanylin, prouroguanylin, cardiolipin, cathepsin, cystatin C, cytochrome c, Fas and Fas ligand (also known as CD95, APO-1), Choline, Caspase-1 (also known as interluekin converting enzyme (ICE)), Creatinine, Atrial Natriuretic Peptide (ANP/N-ANP), and osteoprotegerin (also known as OPG/OCIF).

Markers of Left Ventricular Volume Overload and Myocardial Stretch

Measurement of neurohormones has been explored by the research community for several decades. Biomarkers that have been investigated include the natriuretic peptides, A-type-(ANP), B-type-(BNP), and C-type-(CNP) natriuretic peptide and their N-terminal prohonnones (N-ANP, N-BNP, and N-CNP). ANP (also known as atrial natriuretic peptide) and its inactive form, N-ANP, have been described in, for example, Hall, Eur J Heart Fail, 2001, 3:395-397, which is incorporated by reference in its entirety.

BNP (the active peptide) and N-BNP (the inactive peptide) are found in the circulation. Both peptides are derived from the intact precursor, proBNP, which is released from cardiac myocytes in the left ventricle. Increased production of BNP (or N-BNP; the abbreviation BNP refers to either form of the B-type natriuretic peptide throughout this document) is triggered by myocardial stretch, myocardial tension, and myocardial injury. Studies have demonstrated a positive correlation between circulating levels of BNP, left ventricular volume overload (e.g., left ventricular end diastolic pressure), and an inverse correlation to left ventricular function (e.g., left ventricular ejection fraction and left ventricular mass index).

Measurement of natriuretic peptides, in particular BNP, has been mainly limited to diagnosis of acute decompensation in suspected heart failure patients in the Emergency Department in a hospital setting, providing a prognosis for patients with acute decompensation during hospitalization, and therapy tracking of patients with acute decompensation prior to discharge from hospital. More recent work has investigated the role of BNP during clinic visits and demonstrated that BNP correlates with improvement in the patient's functional status. See, for example, Kohno M; Am J Med. 1995 March; 98(3):257-65, which is incorporated by reference in its entirety. However, testing was infrequent—tests were conducted at baseline, 6 months, and 12 months. Similarly, the study by Kawai (Kawai K; Am Heart J. 2001 June; 141(6):925-32, which is incorporated by reference in its entirety) was limited to testing intervals at baseline, 2 months, and 6 months. Studies by Troughton, Latini and McKelvie also used a testing interval of 4 months or greater (see Lancet 2000, 355:1126-30; Circulation 2002 Nov. 5; 106(19):2454-8; and Circulation 1999 Sep. 7; 100(10):1056-64, respectively, each of which is incorporated by reference in its entirety).

The shortest testing interval was used by Murdoch (Murdoch D R; Am Heart J. 1999 December; 138(6 Pt 1):1126-32, which is incorporated by reference in its entirety). Murdoch used a testing interval of every two weeks, but the study did not consider event detection, safe titration of therapy (by using a GFR marker; see below) or an out-patient or homecare setting.

The only study to have used a higher testing frequency (Braunschweig, F; J Cardiovasc Electrophysiol. 2002 January; 13(1 Suppl):S68-72, which is incorporated by reference in its entirety) investigated the correlation of BNP to weight gain and hemodynamics. A major limitation of this study was the long testing interval of weekly blood draws and again the failure to consider the use of BNP and a GFR marker in a home care setting—in fact, the purpose of the study was to evaluate an implanted hemodynamic sensor and compare this to weight tracking.

There is a danger that the patient or caregiver will drive the patient to a state of under-hydration if they rely on BNP levels alone. Furthermore, a target BNP level for one patient might be unsuitable for another patient because of factors such as age, gender, body mass index, extent of hypertrophy, etc.

In the studies discussed above, the patient and their caregiver did not have access to objective data at a suitable testing interval to allow the prevention of future events (e.g. acute decompensation), rapid drug optimization (e.g. ACE inhibitors, β-blockers, aldosterone receptor blocker), and controlled dose adjustment of diuretics without putting the patient at risk.

Markers of Myocardial Apoptosis or Injury

Markers of myocardial apoptosis provide information on cardiac remodeling, which is an effect of left ventricular volume overload. Measurement of increased myocyte apoptosis arising from excessive myocardial stretch, norepinephrine toxicity, and other proposed mechanisms provide information on cardiac remodeling. Suitable markers include cardiac troponins, including the isoforms troponin I and troponin T (TnI and TnT, respectively), as well as urotensin in all its forms and urotensin-related peptides. Measurement of troponin has traditionally been used to provide a diagnosis of myocardial injury or infarction, distinct from the process of apoptosis. A sensitive immunoassay for a troponin isoform can allow a healthcare provider to obtain information on the extent of myocyte apoptosis and myocyte damage induced by the aforementioned mechanisms or consistent with myocardial ischemia and infarction.

Cardiac troponin levels are frequently above normal values in several disease states in which myocardial necrosis is not a prominent aspect, particularly in pulmonary embolism, heart failure, liver cirrhosis, septic shock, renal failure and arterial hypertension. Sub-clinical myocardial necrosis and increased myocardial apoptosis has been postulated to be the cause of the phenomenon. Increased troponin levels may be the result of ventricular dilatation or hypertrophy. Troponin may act as a marker of myocardial strain, injury, and increased apoptosis (e.g., during acute decompensation or chronic worsening pre-heart failure, heart failure, and hypertension). Apoptosis contributes to myocardiocyte loss in cardiac disease and may have a pathophysiologic role in left ventricular (LV) remodeling. Heart failure is associated with an increase in apoptosis rate and is significantly correlated with parameters of progressive left ventricle remodeling. Low levels of troponin in the circulation correlate with apoptosis rate.

Elevated levels of troponin without elevated levels of creatine kinase is thought to be due to release of troponin from myocardial cells without the disruption of myocardial cell plasma membrane.

Chen measured troponin in the plasma of patients with heart failure. See Chen Y N, Ann Clin Biochem. 1999 July; 36 (Pt 4):433-7, which is incorporated by reference in its entirety. Elevated plasma troponin concentrations were found in 89% of heart failure patients while plasma creatine lcinase-MB (CK-MB) showed no significant difference. During follow-up, serial measurements of cardiac TnI and CK-MB were performed. In heart failure patients, improvement of the clinical profile was associated with declining troponin concentrations, while deterioration of heart function was closely related to increasing troponin concentrations. Cardiac damage relates to functionally overloaded myocytes and troponin may be a sensitive marker both for early detection of myocyte damage and for monitoring of function and prognosis in patients with heart failure. Chen demonstrated that plasma troponin levels that returned to normal in patients whose heart failure was successfully treated had better outcomes than in patients whose troponin remained elevated.

Horwich demonstrated that troponin is elevated in severe heart failure and may predict adverse outcomes (Horwich, T B; Circulation. 2003 Aug. 19; 108(7):833-8, which is incorporated by reference in its entirety). They presented data on 238 patients with advanced heart failure who had troponin assay drawn at the time of initial presentation. Patients with acute myocardial infarction or myocarditis were excluded from analysis. Troponin was detectable (greater than or equal to 0.04 ng/mL) in serum of 117 patients (49.1%). Patients with detectable troponin levels had significantly higher BNP levels and more impaired hemodynamic profiles, including higher pulmonary wedge pressures and lower cardiac indexes. A significant correlation was found between detectable troponin and progressive decline in ejection fraction over time.

Detectable troponin was associated with increased mortality risk. Troponin used in conjunction with BNP improved prognostic value. Therefore, troponin is associated with impaired hemodynamics, elevated BNP levels, and progressive left ventricular dysfunction in patients with heart failure.

Monitoring troponin to detect myocardial infarction in the context of ischemia is already accepted practice (see, for example, Apple FS, European Society of Cardiology and American College of Cardiology guidelines for redefinition of myocardial infarction: how to use existing assays clinically and for clinical trials; Am Heart J. 2002 December; 144(6):981-6, which is incorporated by reference in its entirety).

Therefore, routine measurement of troponin is valuable in the management of the heart failure patient. Serial tracking of troponin will enable information on the patient's condition (whether stable, worsening, or improving) to be determined and will also provide information on future prognosis.

Markers of Inflammation

Inflammation markers can provide information about a patient's condition. A marker of inflammation can be used to predict sudden unexpected death. The marker can be non-specific (i.e., a marker of general inflammation), or specific (i.e., a marker indicating cardiac or vascular inflammation). The marker can be a soluble adhesion molecule (e.g., E-selectin, P-selectin, intracellular adhesion molecule-1, or vascular cell adhesion molecule-1), Nourin-1, a cytokine (e.g., interleukin-1β, -6, -8, and -10 or tumor necrosis factor-alpha), an acute-phase reactants (e.g., hs-CRP), neutrophils, and white blood cell count.

Markers of Anemia

Markers of anemia can also be valuable in tracking heart failure patients. According to one study, heart failure patients with low hematocrits had a significantly higher risk of mortality than those with hematocrit >42% (see Kosiborod, M., et al. Am. J. Med. 2003, 114: 112-119, which is incorporated by reference in its entirety). For example, a hemoglobin level or hematocrit measurement can be used as a marker of anemia.

Markers of Myocardial Ischemia

Markers of myocardial ischemia provide independent information on cardiac output, thrombus formation and embolization, and vascular blood flow. Measurement of such markers (e.g., ischemia-modified albumin, oxygen-regulated peptide (ORP150), free fatty acid, Nourin-1, urotensin in all its forms and urotensin-related peptides, and other known markers) provide an indication of onset of ischemia, magnitude of ischemia, and natural or induced reperfusion.

Markers of Renal Function

The easiest way to measure the glomerular filtration rate (GFR) is with creatinine (Robertshaw M, Lai K N, Swaminatlian R. Br J Clin Pharmacol 1989; 28:275-280, which is incorporated by reference in its entirety). The rate of creatinine addition to the body is proportional to body muscle mass. The rate of creatinine removal is proportional to the concentration in the plasma and the rate of glomerular filtration. For example, a decrease of GFR from 120 mL/min to 60 mL/min would increase the plasma creatinine from 1.0 mg/dL to 2.0 mg/dL. Thus, changes in GFR are mirrored by reciprocal changes in the serum creatinine. Because serum creatinine multiplied by GFR equals the rate of creatinine production, a decrease in the GFR by 50% will cause the serum creatinine to increase by a factor of two at steady-state. Using only a serum or plasma creatinine measurement, the GFR, in mL/min, can be estimated using the formula: GFR=(140−age)×weight (kg)/0.825×plasma creatinine (μmol/L).

Markers of renal function should be monitored regularly in patients on ACE inhibitors, angiotensin II receptor inhibitors, and diuretics. A limited elevation in creatinine level (30 percent or less above baseline) was seen following initiation of therapy with an ACE inhibitor or angiotensin II receptor inhibitors. The increase usually occurred within two weeks of therapy. Regardless of the creatinine value, manifestations of renal failure were not apparent until the GFR was well below 30 mL per minute. Patients with the greatest degree of renal insufficiency experienced the greatest protection from renal disease progression. Hence, upon initiation of an ACE inhibitor or angiotensin II receptor inhibitor, GFR should be monitored, but a decrease is not a reason to withdraw therapy.

The study by Lee (Lee S W; Am J Kidney Dis. 2003 June; 41(6):1257-66, which is incorporated by reference in its entirety) revealed that BNP levels are insensitive to under-hydration in patients on hemodialysis. Lee was evaluating whether BNP might be used to assess hydration status in a patient undergoing aggressive hemodialysis. When these findings are applied to the process of diuresis using either intravenous or oral diuretic therapy, one would realize that BNP cannot be used to detect a state of over-diuresis which could be life threatening. Consequently, routine measurement of a glomerular filtration rate marker is necessary to determine whether the patient is at risk of under-hydration through over-use of diuretic therapy.

Several biochemical methods exist for the measurement of GFR. Generally, these measure the level of an analyte that is metabolized at a constant rate, so that an increase in circulating levels of the analyte indicates renal failure. Suitable such analytes include creatinine and Cystatin C. See, for example, Newman, D J, Ann Clin Biochem. 2002 March; 39(Pt 2):89-104; and Perrone R D et al, Clin Chem. 1992 October; 38(10):1933-53:, each of which is incorporated by reference in its entirety. Measurement of GFR with creatinine (plasma or serum creatinine) can be achieved with the Cockroft and Gault equation to adjust for age, weight, and gender.

An alternative measurement of GFR can be achieved with Cystatin C. Cystatin C has a low molecular weight and is filtered freely at the glomerular membrane. Cystatin C has been proposed as an alternative and superior marker to serum creatinine. Cystatin C is produced by all nucleated cells and catabolized by renal tubular cells. Its rate of production is constant and is not affected by muscle mass, inflammation, and it does not have a circadian rhythm.

Cystatin C was found to be more specific than serum creatinine in evaluating renal function with a tighter distribution of values around the regression line (Mussap, M; Kidney International, Vol 61 (2001), pp 1453-1461, which is incorporated by reference in its entirety). Mussap also reported that Cystatin C rises earlier and more rapidly than serum creatinine as GFR decreases—it has higher sensitivity than both serum creatinine and GFR derived from the Cockroft-Gault equation. commercial test for serum Cystatin C is available from Dade Behring (nephelometric assay; N-latex Cystatin C Assay; 6 minute test).

Markers of Electrolyte Balance

Electrolyte balance is the condition where a patient's electrolytes (for example, soluble ions such as Na⁺ and K⁺) are in the normal concentration range. The subject may be a heart failure patient with a stable condition, a heart failure patient with an unstable condition, a patient with mild, moderate, or advanced hypertension, or a patient with recent myocardial infarction. Typical values of normal fluid and electrolyte balance are as follows and are dependent upon the age and sex of the individual: for an average 70 kg man the total body water is typically 42 L (˜60% of body weight), with 28 L being in the intracellular and 14 L in the extracellular compartments. The plasma volume is 3 L and the extravascular volume is 11 L. Total body Na⁺ is typically 4200 mmol (50% in extracellular fluid, (ECF)) and the total body K⁺ is typically 3500 mmol (about 50-60 mmol in ECF). The normal osmolality of ECF is 280-295 mosmol/kg.

Hypokalemia is a common adverse effect of diuretic therapy and may also increase the risk of digitalis toxicity. Hence, plasma or serum potassium levels should be routinely measured in heart failure patients in order to avoid such undesirable side effects. Potassium is typically measured using an ion-selective electrode (e.g. i-STAT, i-STAT Corp.)

Markers of Sodium Retention

Markers of sodium retention or excessive sodium intake can provide an estimate of sodium retention, electrolyte balance, and sodium consumption. One suitable marker is uroguanylin, which is an intestinal natriuretic hormone and functions as an endocrine modulator of sodium homeostasis. In a patient with congestive heart failure, levels of uroguanylin measured in urine are known to be substantially higher than in controls. The increased urinary uroguanylin excretion in patients with heart failure may be an adaptive response. The urinary excretion of uroguanylin is significantly higher in the presence of a high salt diet and significantly correlated with urinary sodium. Measurement of uroguanylin can provide unique information on sodium homeostasis and the patient's status. Such measurement may be used to make decisions on intake of fluid and sodium to avoid adverse events.

Diagnostic Device

A diagnostic device enables a patient or health care provider to measure the level of a biomarker, or a panel of biomarkers. The device can measure and record the levels of one or more biomarkers, record patient input regarding signs and symptoms of disease, provide feedback to the patient, and provide recorded results to a health care provider.

Using the device, the patient's condition can optionally be monitored remote from a dedicated health care facility, such as doctor's office or hospital. The device can help the health care provider measure the effectiveness of both pharmacological and non-pharmacological aspects of a care plan, and to monitor the progression of the disease. The device can also aid in assessing the patient's compliance to therapy and future prognosis.

The biomarkers measured by the device can include a marker of left ventricular volume overload or myocardial stretch, a marker of myocardial apoptosis or injury, a marker of myocardial ischemia, a marker of inflammation, a marker of anemia, a marker of renal function, a marker of electrolyte balance, or a marker of sodium retention. In addition, the device can include probes for measuring the patient's vital signs, such as weight, temperature, heart rate, variability of heart rate, breathing rate, blood pressure, and blood oxygen saturation (measured, for example, by pulse oximetry). The device can record electrical measurements, such as an electrocardiogram, from the patient. The device can present queries to the patient and record the patient's responses. The queries can relate to the patient's condition, such as whether the patient is suffering any symptoms or when medication was taken.

The patient can use the device on a regular basis, or as instructed by a caregiver. For example, the patient may use the device daily, every other day, weekly, or on another appropriate interval. Under certain circumstances, fewer than all available tests will be performed. For example, a patient may perform a blood pressure measurement on a daily basis, but measure a marker of left ventricular volume overload or myocardial stretch on a weekly basis. Based on the results of the tests, the device can respond with instructions for the patient. The instructions can be configured based on a treatment algorithm. The algorithm can be adjusted to suit the needs of the patient. For example, if a health care provider can enter information specific to a particular patient (such as a threshold value for a biomarker) into the device.

The biomarkers can be measured in a sample. The sample is taken from the patient and can be a sample of blood, plasma, serum, saliva or urine. In one embodiment, the sample is a blood sample. Such a sample may be taken by the patient by, for example, collecting a blood sample having a volume of less than one microliter up to a volume of several hundred microliters following puncture of the skin with an appropriate lancing device. The biomarkers monitored can be detected using, for example, an immunoassay, a biosensor, an ion-selective electrode, or another suitable technology.

For example, the markers can be detected using an immunoassay. An immunoassay is performed by contacting a sample from a subject to be tested with an appropriate antibody under conditions such that immunospecific binding can occur if the marker is present, and detecting or measuring the amount of any immunospecific binding by the antibody. Any suitable immunoassay can be used, including, without limitation, competitive and non-competitive immunoassay systems or ligand-binding systems known to one skilled in the art.

For example, a marker can be detected in a fluid sample by means of a one-step sandwich assay. A capture reagent (e.g., an anti-marker antibody) is used to capture the marker. Simultaneously, a directly or indirectly labeled detection reagent is used to detect the captured marker. In one embodiment, the detection reagent is an antibody. Such all immunoassay or another design known to one skilled in the art can be used to measure the level of an aforementioned biomarker in an appropriate body fluid.

A GFR marker (e.g. serum creatinine) can be measured using a biosensor, an enzymatic assay, or amperometrically. See, for example, Erlenkotter A, Anal Bioanal Chem. 2002 January; 372(2):284-92; Leger F, Eur J Cancer. 2002 January; 38(1):52-6; and Tombach B, Clin Chim Acta. 2001 October; 312(1-2):129-34, each of which is incorporated by reference in its entirety.

The measurement of a biomarker by both immunoassay and biosensor (e.g. calorimetrically) has been demonstrated by Metrika with their patented MODM™ (Micro Optical Detection Method) technology. This integrates miniaturized digital electronics, micro-optics and solid-state chemistries into an easy to use, low-cost, single-use instrument. MODM technology is designed for simultaneous measurement of immunodiagnostic and general chemistries in less than ten minutes. Ostex International Inc. has used the same technology to develop the OSTEOMARK NTx Point-of-Care (POC). This is a disposable single use device that provides a normalized measurement of the bone marker ‘NTx’ by measuring NTx and creatinine levels in a sample and then calculating the ratio result. The POC is intended for use in a physician's office and takes 5 minutes to process.

The device can be included in a diagnostic kit, which can optionally include one or more of the following: instructions for using the kit for event detection, diagnosis, prognosis, screening, therapeutic monitoring or any combination of these; a disposable testing cartridge containing the necessary reagents to conduct a test; or an instrument or device that measures the result of biomarker testing and optionally, allows manual or automatic input of other parameters, storage of said parameters, and evaluation of said parameters alongside or separate from the evaluation of the measured biomarkers.

The testing cartridge or cartridges supplied in the kit allow the user to measure one or more biomarkers, such as a marker of left ventricular volume overload or myocardial stretch and optionally a measurement of a marker of renal function, a measurement of a marker of myocardial apoptosis, a measurement of a marker of myocardial ischemia, a measurement of a marker of myocardial injury, a measurement of a marker of anemia, a measurement of a marker of electrolyte balance, and a marker of sodium retention.

The testing cartridge or testing cartridges allow the sequential or serial measurement of a marker of left ventricular volume overload or myocardial stretch and a marker of renal function.

The testing cartridge or testing cartridges allow the sequential or serial measurement of a marker of left ventricular volume overload or myocardial stretch, a measurement of a marker of renal function, a measurement of a marker of myocardial apoptosis or injury, a measurement of a marker of myocardial ischemia, a measurement of a marker of inflammation, a measurement of a marker of anemia, a measurement of a marker of electrolyte balance, and a marker of sodium retention. A combination cartridge can test two or more different markers from a single sample.

The instrument (durable or disposable) can measure the result of biomarker testing and optionally, allows manual or automatic input of other parameters, storage of said parameters, and evaluation of said parameters with or separate to the measured biomarkers.

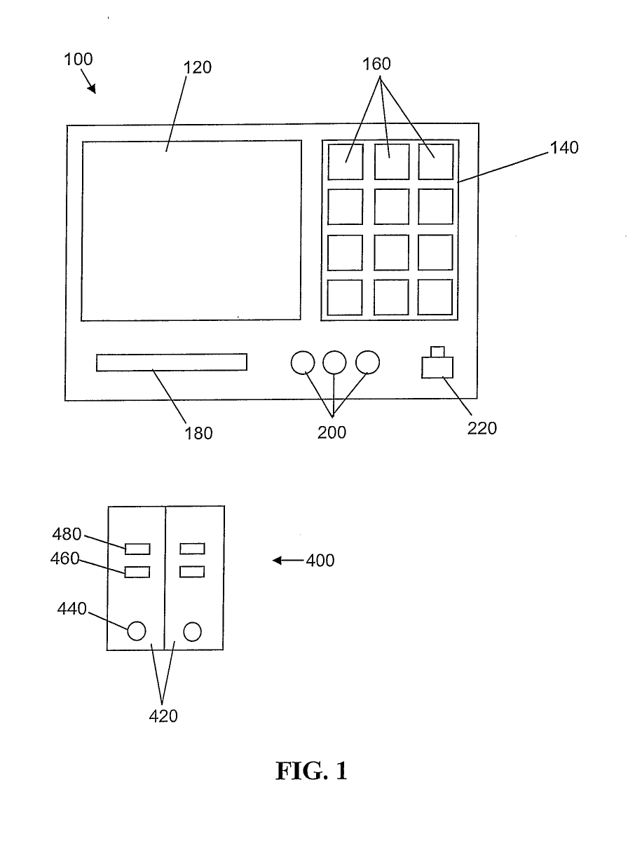

Referring to FIG. 1, diagnostic device 100 includes display 120 and input region 140. The display 120 may be used to display images in various formats, for example, joint photographic experts group (JPEG) format, tagged image file format (TIFF), graphics interchange format (GIF), or bitmap. Display 120 can also be used to display text messages, help messages, instructions, queries, test results, and various information to patients. In some implementations, display 120 supports the hypertext markup language (HTML) format such that displayed text may include hyperlinks to additional information, images, or formatted text. Display 120 can further provide a mechanism for displaying videos stored, for example in the moving picture experts group (MPEG) format, Apple's QuickTime format, or DVD format. Display 120 can additionally include an audio source (e.g., a speaker) to produce audible instructions, sounds, music, and the like.

Input region 140 can include keys 160. In one embodiment, input region 140 can be implemented as symbols displayed on the display 120, for example when display 120 is a touch-sensitive screen. Patient instructions and queries are presented to the patient on display 120. The patient can respond to the queries via the input region.

Device 100 also includes cartridge reader 180, which accepts diagnostic test cartridges for reading. The cartridge reader 180 measures the level of a biomarker based on, for example, the magnitude of a color change that occurs on a test cartridge 400. Device 100 also includes probe connections 200, which connect probes (e.g., a probe of weight, temperature, heart rate, variability of heart rate, breathing rate, blood pressure, or blood oxygen saturation) to the device.

Device 100 further includes a communication port 220. Communication port 220 can be, for example, a connection to a telephone line or computer network. Device 100 can communicate the results of patient tests to a health care provider from a remote location. Likewise, the health care provider can communicate with the device 100 (e.g., to access stored test results, to adjust device parameters, or send a message to the patient).

Cartridge 400 is shown with two testing zones 420. In general, a cartridge can include 1, 2, 3, 4, or 5 or more testing zones. Each testing zone 420 can test the level of a biomarker. Each testing zone 420 includes a sample input 440, a control result window 460 and a test result window 480. In one embodiment, the cartridge 400 is an immunochromatographic test cartridge. Examples of immunochromatographic tests and test result readers can be found in, for example, U.S. Pat. Nos. 5,504,013; 5,622,871; 6,235,241; and 6,399,398, each of which is incorporated by reference in its entirety.

A patient can use device 100 for testing and recording the levels of various biomarkers that provide information about the patient's health. Various implementations of diagnostic device 100 may access programs and/or data stored on a storage medium (e.g., video cassette recorder (VCR) tape or digital video disc (DVD); compact disc (CD); or floppy disk). Additionally, various implementations may access programs and/or data accessed stored on another computer system through a communication medium including a direct cable connection, a computer network, a wireless network, a satellite network, or the like.

The software controlling the diagnostic device and providing patient feedback can be in the form of a software application running on any processing device, such as, a general-purpose computing device, a personal digital assistant (PDA), a special-purpose computing device, a laptop computer, a handheld computer, or a network appliance.

A diagnostic device may be implemented using a hardware configuration including a processor, one or more input devices, one or more output devices, a computer-readable medium, and a computer memory device. The processor may be implemented using any computer processing device, such as, a general-purpose microprocessor or an application-specific integrated circuit (ASIC). The processor can be integrated with input/output (I/O) devices to provide a mechanism to receive sensor data and/or input data and to provide a mechanism to display or otherwise output queries and results to a service technician. Input device may include, for example, one or more of the following: a mouse, a keyboard, a touch-screen display, a button, a sensor, and a counter.

The display 120 may be implemented using any output technology, including a liquid crystal display (LCD), a television, a printer, and a light emitting diode (LED). The computer-readable medium provides a mechanism for storing programs and data either on a fixed or removable medium. The computer-readable medium may be implemented using a conventional computer hard drive, or other removable medium such as those described above with reference to. Finally, the system uses a computer memory device, such as a random access memory (RAM), to assist in operating the diagnostic device.

Implementations of a diagnostic device can include software that directs the patient in using the device, stores the result of biomarker measurements, determines whether a tested biomarker level requires medical attention for the patient, instructs the patient in adjusting or maintaining therapy, and communicates the patient's information to his or her caregiver. Patients suffering from, for example, heart failure or hypertension, or patients at risk of a myocardial infarction can use the device.

The device 100 can provide access to applications such as a medical records database or other systems used in the care of patients. In one example, the device connects to a medical records database via communication port 220. Device 100 may also have the ability to go online, integrating existing databases and linking other websites. Online access may also provide remote, online access by patients to medical information, and by caregivers to up-to-date test results reflecting the health of patients.

The device can be used in the hospital, physician's office, clinic, and patient's home either by the patient or an attendant care giver. In one embodiment, the invention is practiced in the patient's home allowing the patient to be monitored, his or her therapy optimized, and adverse events that require hospitalization to be avoided.

Decision Points

The device can be configured to respond to the measured level of a biomarker, in particular when the level of the biomarker indicates a change in the patient's health status. For example, the device can be configured to store the results of tests and determine changes in the levels of markers over time. A change in results over time can be an acute change or a chronic change. An acute change can be a significant change in the level of a biomarker over a short period of time. The magnitude of change and period of time can be different for each biomarker. The device can be configured to compare each new test result either to a stored values of recent test results (e.g., the previous 1, 2, 3, 4, 5 or more results), or to an aggregate measure of recent test results (such as an average) to determine if an acute change has occurred. In one example, an acute change is detected by the percentage change in a test result from the previous result.

Chronic changes can be detected as well. A chronic change can be a change in the level of a biomarker that occurs over a long period of time. For example, a chronic change can occur such that many testing intervals pass without an acute change being detected, yet the level of biomarker is significantly different. To detect a chronic change, the device can compare the results of each new test to a stored result of an earlier test, or to an aggregate measure of earlier tests. For detecting chronic changes, the earlier test can be, for example, 4-12 weeks prior to the new test result. In one example, the aggregate measure can be a rolling average, such as a 4-week, 8-week, or 12-week rolling average.

The device can also be configured to compare test results to a stored threshold value or range. The threshold value can be an upper or lower limit or range of values. Thus, the device can determine if the measured value of a marker, or group of markers, is a safe level, a dangerous level, or indicates an emergency. The device can alert the patient to the results of the test and can be configured, when appropriate to instruct the patient to seek medical care.

The device can also be configured to track combinations of markers, for example, an average value of two markers, the difference in level between two markers, a ratio of the levels of two markers, or whether two or more markers exceed their respective threshold values at the same time. The device can be configured to track one or more markers in combination with a patient's signs and symptoms.

The device can be personalized for a patient. The threshold values and other parameters for each biomarker can be adjusted (for example, by a physician or other caregiver) based on the circumstances of the patient, such as, for example, age, gender, or disease status. The questions and responses that the device presents to the patient can also be adjusted.

An individual patient's test results can be used to assign the patient to a category of patients. Exemplary categories include responders and non-responders, or low-, medium-, and high-risk for side effects. The results can be used to select a type of therapy for a patient, for example, to indicate that a patient should be treated with a member of a particular class of drugs (such as, for example, an ACE inhibitor, or a statin). In some circumstances, the results can be used to suggest a particular member of a family of drugs for a patient, e.g., a particular statin. Reviewing stored test results can help a patient (or the patient's health care provider) keep track of therapeutic progress and safety.

In some embodiments, tests are designed to measure aspects of a patient's physiology. The physiological measurement can be indirect, i.e., a marker is used to report on the patient's physiological state. Some physiological states can be measured by more than one marker. For example, left ventricular volume overload and myocardial stretch can be measured by the marker BNP. Creatinine and cystatin C are markers for renal function. A number of markers of myocardial apoptosis or injury exist; carciac troponins are one example. Inflammation can also be measured by a number of markers, including, for example, E-selectin, P-selectin, intracellular adhesion molecule-1, vascular cell adhesion molecule-1, Nourin-1, interleukin-1β, interleukin-6, interleukin-8, interleukin-10, tumor necrosis factor-alpha, hs-CRP, neutrophils, or white blood cell count. Hemoglobin or hematocrit measurements can be used to detect anemia. Myocardial ischemia can be measured by markers including ischemia modified albumin, oxygen-regulated peptide, and free fatty acid. Electrolytes and sodium concentration can also be measured.

Other embodiments are within the scope of the following claims.

What is claimed is:

1. A method of predicting patient response to a drug or drug candidate, comprising:
obtaining a plurality of test results for a plurality of patients, wherein the plurality of test results include, for each patient, at least one result of a biochemical test, a medical test, or a genetic test; and at least one result of a test of patient response to the drug or drug candidate; and
detecting a correlation between the result of a biochemical test, a medical test, or a genetic test and the result of the test of patient response to the drug.

2. The method of claim 1, wherein the test of patient response to the drug or drug candidate is a test of drug efficacy.

3. The method of claim 1, wherein the test of patient response to the drug or drug candidate is a test of drug safety.

4. The method of claim 1, wherein the plurality of test results includes, for each patient, at least one result of a plurality of biochemical tests.

5. The method of claim 4, wherein at least one biochemical test includes an immunoassay.

6. The method of claim 1, further comprising obtaining at least one result of a biochemical test, a medical test, or a genetic test, from a prospective patient; and predicting a response of the prospective patient based on the result obtained for the prospective patient and the detected correlation.

7. The method of claim 6, wherein obtaining at least one result from the prospective patient includes obtaining at least one result of a plurality of biochemical tests from the prospective patient.

8. The method of claim 7, wherein at least one biochemical test includes an immunoassay.

9. The method of claim 4, wherein the plurality of test results include, for each patient, a plurality of results of a plurality of biochemical tests.

10. A method of monitoring a patient comprising:
administering a drug or drug candidate to a patient;
obtaining at least one result of a biochemical test, a medical test, or a genetic test from the patient; and
correlating at least one result with at least one measure of patient response.

11. The method of claim 10, wherein the measure of patient response is a measure of drug or drug candidate efficacy.

12. The method of claim 10, wherein the measure of patient response is a measure of drug or drug candidate safety.

13. The method of claim 10, wherein obtaining at least one result from the patient includes obtaining at least one result of a plurality of biochemical tests from the patient.

14. The method of claim 13, wherein at least one biochemical test includes an immunoassay.

15. The method of claim 13, wherein at least one result of the plurality of biochemical tests correlates with a measure of drug or drug candidate efficacy.

16. The method of claim 13, wherein at least one result of the plurality of biochemical tests correlates with a measure of drug or drug candidate safety.

17. The method of claim 15, wherein at least one result of the plurality of biochemical tests correlates with a measure of drug or drug candidate safety.

18. The method of claim 13, wherein obtaining at least one result from the patient includes obtaining a plurality of results of a plurality of biochemical tests from the patient.

19. The method of claim 10, further comprising altering the administering of the drug or drug candidate to the patient based on the obtained result.

20. The method of claim 10, wherein the drug or drug candidate is a statin.

21. The method of claim 20, wherein obtaining at least one result includes obtaining a result of a test for a marker of endothelial dysfunction, a marker of inflammation, a marker of plaque stability, a marker of systolic function, a marker of safety, or a combination thereof.

22. A method, comprising:
obtaining a result of at least one of a biochemical test, a medical test, and a genetic test performed on a human subject,
referencing a correlation between the result of the at least one biochemical test, medical test, and genetic test and a human response to a drug, the correlation having been determined based on a result of the at least one biochemical test, medical test, and genetic test obtained from each of multiple reference human subjects and a response of each of the multiple human subjects to a drug, and
predicting a response of the human subject to the drug based on the result of the at least one biochemical test, medical test, and genetic test and human response and the correlation.
